(19) United States
(12) Patent Application Publication
(10) Pub. No.: US 2025/0111242 A1
(43) Pub. Date: Apr. 3, 2025

(54) INFORMATION EXCHANGE PLATFORM USING REINFORCEMENT LEARNING MODELS

(71) Applicant: inpharmD, Atlanta, GA (US)

(72) Inventors: Tulasee Rao CHINTHA, Atlanta, GA (US); Ashish Advani, Atlanta, GA (US); Steve Pickette, Atlanta, GA (US)

(21) Appl. No.: 18/904,930

(22) Filed: Oct. 2, 2024

Related U.S. Application Data
(60) Provisional application No. 63/587,239, filed on Oct. 2, 2023.
(60) Provisional application No. 63/667,318, filed on Jul. 3, 2024.

(51) Int. Cl. G06N 3/092; G16H 70/40

(52) U.S. Cl. CPC G06N 3/092; G16H 70/40

(57) ABSTRACT

Various systems and methods providing a platform that facilitates creating, managing, and searching for documents, such as medical documents, and the evaluation of medical workflows, are described. In some embodiments, the systems and methods utilize machine learning models (e.g., large language models, or LLMs), such as ML models that employ reinforcement learning from human feedback (RLHF), or similar reinforcement learning models, to enhance and/or optimize operations and processes provided or supported by the platform, such as search queries, scenario, generation, and information retrieval operations, and so on.

### CROSS REFERENCE TO RELATED APPLICATIONS

This application claims priority to U.S. Provisional Patent Application No. 63/587,239, filed on Oct. 2, 2023, entitled INFORMATION EXCHANGE PLATFORM USING REINFORCEMENT LEARNING MODELS, and U.S. Provisional Patent Application No. 63/667,318, filed on Jul. 3, 2024, entitled INFORMATION EXCHANGE PLATFORM USING REINFORCEMENT LEARNING MODELS, which are hereby incorporated by reference in their entirety.

### BACKGROUND

While online searching has become ubiquitous, current search engines and information portals are often unable to provide information that sufficiently answers questions, such as questions that lead to complex or imprecise answers. The medical field can be particularly cumbersome because information may be scattered across different and opaque corpuses of medical papers, clinical information, study results, drug/medicine information, drug utilization, insurance claims, and other data stores of information. Further, there are many stakeholders (e.g., doctors, patients, drug manufacturers, device and/or equipment providers) that seek different types of information or solutions.

### BRIEF DESCRIPTION OF THE DRAWINGS

Embodiments of the present technology will be described and explained through the use of the accompanying drawings.

FIG. 1 is a block diagram illustrating a suitable network environment that supports a document management platform.

FIG. 2A is a block diagram illustrating example modules of a formulary system.

FIG. 2B is a diagram illustrating various components of the evaluation system.

FIGS. 2C-2E are diagrams illustrating example user interfaces presented by a review dashboard.

FIG. 2F is a flow diagram illustrating a method for perform an action associated with a formulary.

FIG. 3 is a diagram illustrating data flows between components of the information exchange platform.

FIG. 4 is a flow diagram illustrating an example method for determining drug shortages using the platform.

FIGS. 5A-5S are diagrams illustrating details associated with determining drug shortages using the platform.

FIGS. 6A-6B are diagrams illustrating a dashboard and various associated information flows provided by the platform.

FIGS. 7A-7L are diagrams illustrating details of the information exchange platform.

FIGS. 8A-8E are diagrams illustrating details of a user experience via the information exchange platform.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

Various systems and methods providing a platform that facilitates creating, managing, and searching for documents, such as medical documents, and the evaluation of medical workflows, are described. In some embodiments, the systems and methods utilize machine learning models (e.g., large language models, or LLMs), such as ML models that employ reinforcement learning from human feedback (RLHF), or similar reinforcement learning models, to enhance and/or optimize operations and processes provided or supported by the platform, such as search queries, scenario, generation, and information retrieval operations.

In some embodiments, the platform utilizes, provides, or supports a formulary system, where a formulary acts as a data object or data structure (and along with an access API) via which the platform may perform actions with respect to queries for documents or information, drugs or other pharmaceuticals, and so on.

In some embodiments, the platform provides access to information based on organizational structure, such as an internal hospital, external business partners, external patients, and so on. The platform may enable formulary modifications based on the structure and depending on access levels, certification privileges, or other factors.

In some embodiments, the platform can determine or predict drug scarcities, using characteristic information for drugs medicines and learning models to predict or estimate future or potential drug shortages or scarcities. The platform may publish or present a dashboard for various users or entities, which presents, in real-time, a risk status or probability of scarcity for drugs, medicines, and other medical substances.

In some embodiments, the platform may include an evaluation system, which may evaluate medical workflows to optimize performance parameters (e.g., health outcomes, clinical efficacy, costs, revenues, information access for medical staff, patient engagement, alternative medications during regular use and/or clinical trials, classify and/or cluster patients and service providers) based on predetermined criteria.

Thus, in some embodiments, the platform enables or provides quality, relevancy, and/or scalability of a formulary and related databases. Further, the platform enables evaluation of individual and institutional contributions, submission of queries and new evidence, provision of answers to users, provision of recommendations, captured outcomes of the recommendation, and so on.

The platform, in various aspects and implementations, can support document management operations, information exchange operations, search queries and requests, request resolutions, and so on, within clinical or healthcare settings. The platform may act as a Reinforcement Learning-Based Pharmacy Q&A System with LLM Integration, an AI-Driven Pharmacy Q&A System with Reinforcement Learning Models, a Reinforcement Learning-Driven Solution for Efficient Communication and Request Management in Healthcare Settings, a Reinforcement Learning-Enabled Intelligent Healthcare Communication and Request Management System, and other iterations or focused services.

Various embodiments of the system, methods, and/or platform will now be described. The following description provides specific details for a thorough understanding and an enabling description of these embodiments. One skilled in the art will understand, however, that these embodiments may be practiced without many of these details. Additionally, some well-known structures or functions may not be shown or described in detail, so as to avoid unnecessarily obscuring the relevant description of the various embodiments. The terminology used in the description presented below is intended to be interpreted in its broadest reasonable manner, even though it is being used in conjunction with a detailed description of certain specific embodiments.

### Examples of a Suitable Network Environment

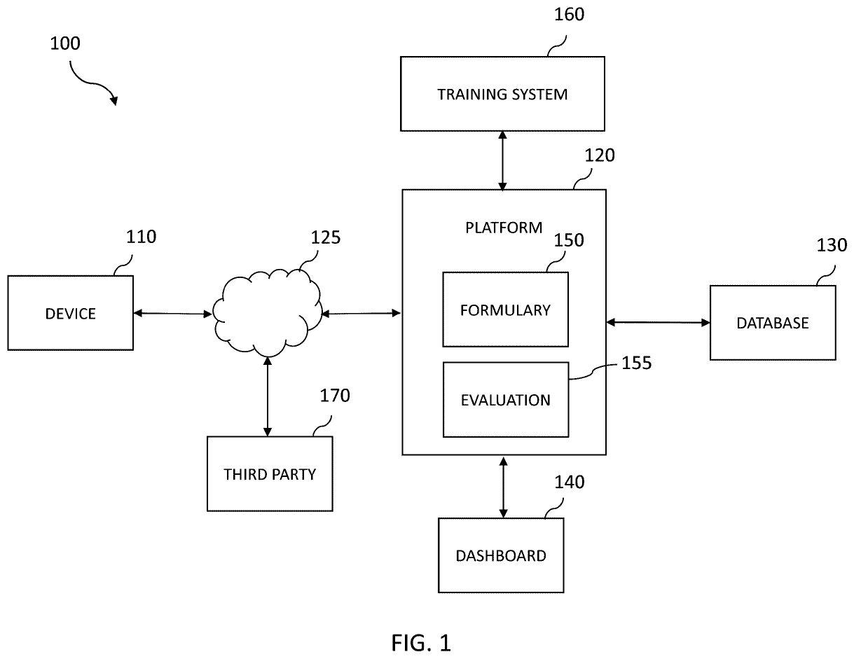

The technology described herein is directed, in some embodiments, to a platform that utilizes machine learning (ML) models, such as reinforcement learning (RL) models, to optimize and/or enhance actions performed by the platform (e.g., searching for and retrieving medical information in response to clinical questions), user experiences in obtaining information via the platform, and so on. FIG. 1 is a block diagram illustrating a suitable network environment 100 that supports an information exchange platform 120.

A user device, such as a device associated with a doctor, patient, clinician, drug manufacturing representative, or other interested parties, can transmit queries, searches, or other information to the platform 120 over a network 125, such as the Internet. For example, the device 110 can be a mobile device or laptop that supports a mobile application or web page via which the user can submit or input such queries.

The platform 120 is associated with one or more databases 130 or data stores, such as databases 130 that store information associated with retrieved or cached documents, previous query information, previous results or action information, and so on. The databases 130 may be part of the platform 120 and/or be associated with the platform 120 but located remote from the platform 120.

The platform 120, in some embodiments, provides and supports a dashboard 140 via which information associated with results of queries by the device 110 can be published or presented to users. For example, a doctor may access the dashboard 140 using their device 110 via the platform 120, and utilize the dashboard to submit a query, view and analyze results, provide input to cause actions to be performed, update or refine queries, and so on. Further, the dashboard may enable a user to submit a query or clinical evidence, receive or obtain answers or information evaluate health or treatment scenarios, receive recommendations, submit outcomes of implemented recommendations, and so on.

In some embodiments, the dashboard 140 enables the user to initiate alternative treatment scenarios, register or request updates, compare outcomes from formulary modification actions, and so on.

As described herein, the platform 120 utilizes training models, such as RL or other ML models (e.g., LLMs), to update, enhance or optimize its performance and/or operations. The platform 120 can include or access a training system 160, which maintains and provides such models. For example, the models can include reinforcement learning models (e.g., RLHF) and other models that act to train reward models using human feedback or preferences to optimize policies/operations via optimization techniques. The training system 160 can include other ML or AI models employed to enhance or optimize the learning and operations of the platform 120.

In some embodiments, the platform 120 includes a formulary system 150, which can include or store formularies as data structures or actionable data objects. A formulary, in some cases, is a list of medicines or drugs that can be associated with a prescription. Thus, the formulary system 150 may maintain a data structure that relates prescriptions to drugs or medicines, mapping the drugs/medicines to prescriptions and other medical instructions or treatments (e.g., based on monitoring and outcome reporting protocols).

In some embodiments, the platform 120 includes an evaluation system 155, such as a system that performs economic evaluations associated with impacts or outcomes based on changes/decisions to formularies (e.g., data stored by the formulary system 150). As described herein, the evaluation system 155 may operate to evaluate various medications/drugs/devices/staff available for a certain use are interchangeable, and determine, based on various analyses, whether to substitute or change out one medication for another.

In some embodiments, the evaluation system 155 performs techniques that receive data from disparate sources (e.g., different hospital systems, trials, medical device applications), such as large, noisy sets of data. The system 155 may apply AI/ML (e.g., certain transformers) to clean up the data, generate, store, and run certain scenarios that generate recommendations and/or identify medications that are interchangeable to other medications and/or therapies. For example, the system 155 attempts to identify medications within the noisy data sets that are interchangeable based on prior applications or contexts and recommend certain medications (from interchangeable sets or groups) that have certain current or predicted characteristics (e.g., lower prices, earlier availability, clinical efficiency, adherence, complications/side effects, resource utilization, and so on).

The system 155, therefore, may act as a tool of opportunity that reviews the use or application of medications (e.g., or other medical entities, such as devices, objects, and so on) with large, uncorrelated data sets and identifies the opportunities (e.g., interchange possibilities). These evidence-based determinations enable the system 155, or an entity employing the system 155, to identify therapeutically interchangeable medications (or therapy alternatives) and perform actions based on those identifications.

In some embodiments, the system 155 may present certain recommendations via a dashboard that presents comparisons, potential savings or availabilities, and so on. The dashboard may sort or filter medications by various associated metadata (e.g., cost savings, reimbursement income, available amounts, and so on), and/or surface medications that satisfy one or more requests (e.g., “present the lowest cost therapeutically interchangeable medication for XXXXX application”).

In some embodiments, the system 155 tracks execution of alternative scenarios and attribute improvements in outcomes to a specific scenario. In some embodiments, the system 155 allocates a share of improvements to a third party (e.g., a service provider, supplier, drug manufacturer, and so on).

In some embodiments, the platform enables users to set the share manually. In some embodiments, the share is set according to an electronic (smart) contract.

In some embodiments, a medical workflow and its elements are represented by a vector that can be operated upon by various ML techniques. The vector representation may be validated to reflect reliable multi-dimensional data downloaded from a database or cleaned up by other ML techniques.

The platform 120 can access one or more third party sites 170 via the network 125. For example, the third-party sites may include document databases that contain academic papers, clinical studies, and so on, drug or medicine information, drug manufacturer information, global weather information, and so on.

Further, in some embodiments, the platform 120 may communicate with third party entities or devices, such as databases, entity systems, and so on. The platform 120 may may access and/or receive information from drug databases, chart databases, intervention databases, and so on. For example, upon receipt of the information from third parties, the platform 120 analyzes the information for quality, consistency, and/or relevance. Once the information is verified, it is integrated into the formulary 150 to reflect specific drug properties, patient outcomes, pricing options, distribution channels, shortages, and so on.

Further details regarding the platform 120 and the associated components depicted in FIG. 1 are described herein.

FIG. 1 and the components, systems, servers, and devices depicted herein provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, magnetic or optical cards, flash memory, or other types of media/machine-readable medium suitable for storing electronic instructions.

The network 125, or cloud, can be any network, ranging from a wired or wireless local area network (LAN), to a wired or wireless wide area network (WAN), to the Internet or some other public or private network, to a cellular system or network (e.g., 4G, LTE, or 5G network), and so on. Further, the user device 110 can be any computing device and/or mobile device, including a smart phone, tablet, laptop, desktop computer, gaming device, and so on. While the connections between the various devices and the network 120 and are shown as separate connections, these connections can be any kind of local, wide area, wired, or wireless network, public or private.

Further, any or all components depicted in the Figures described herein can be supported and/or implemented via one or more computing systems, services (e.g. cloud instances), or servers. Although not required, aspects of the various components or systems are described in the general context of computer-executable instructions, such as routines executed by a general-purpose computer, e.g., mobile device, a server computer, a cloud computer or service, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, cloud-based systems, microprocessor-based or programmable consumer electronics, set-top boxes, network PCs, mini-computers, mainframe computers, AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Personal Area Network (PAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as an exercise machine, display device, or mobile or portable device, and thus, while certain hardware platforms are described herein, aspects of the system are equally applicable to nodes on a network. In some cases, the mobile device or portable device may represent the server portion, while the server may represent the client portion.

### Examples of the Information Exchange Platform

As described herein, in some embodiments, the platform 120 can manage and optimize the searching for and retrieval of medical/clinical information for various types of queries. The platform 120 provides the formulary system 150, which can function as an actionable data object that relates various types of information, such as drug information with prescription information, trial information, study information, and so on.

Thus, the platform 120 may ensure quality, relevancy, and/or scalability of the formulary and related databases, and enable addition, modification, and deletion of facts, rules and user feedback. Further, it enables evaluation of individual and institutional contributions, communication of queries and new evidence, provides answers to users, provides recommendations and captures outcomes of the recommendations.

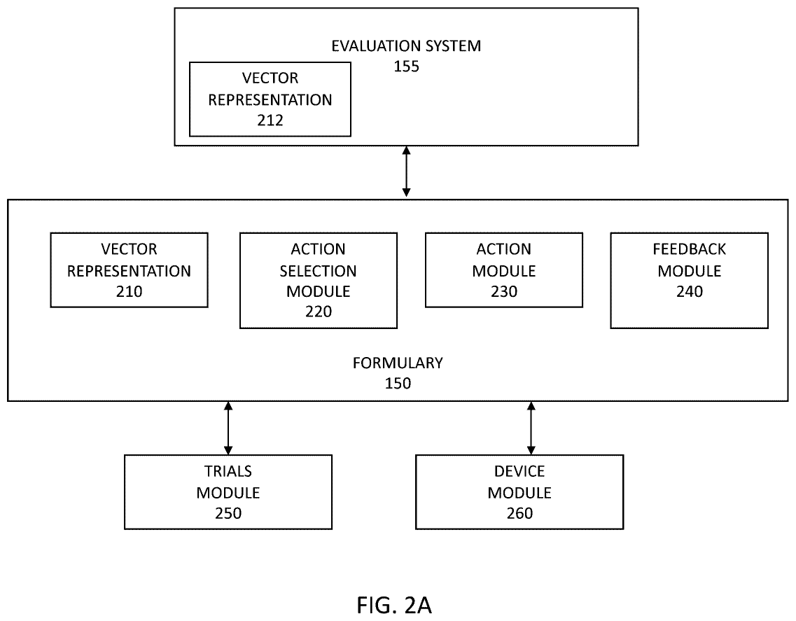

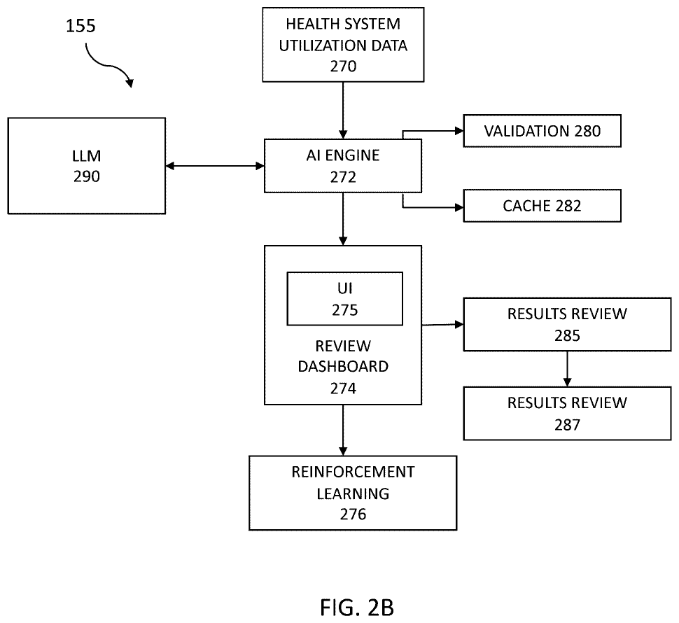

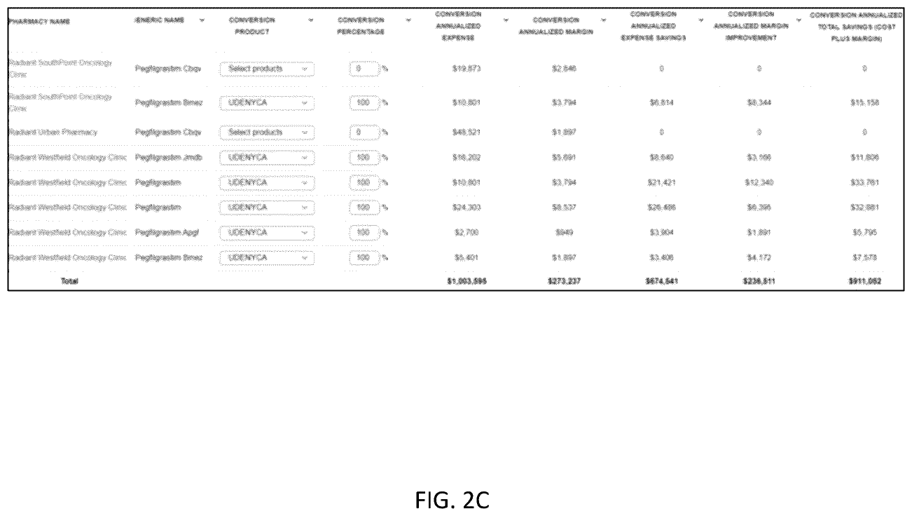

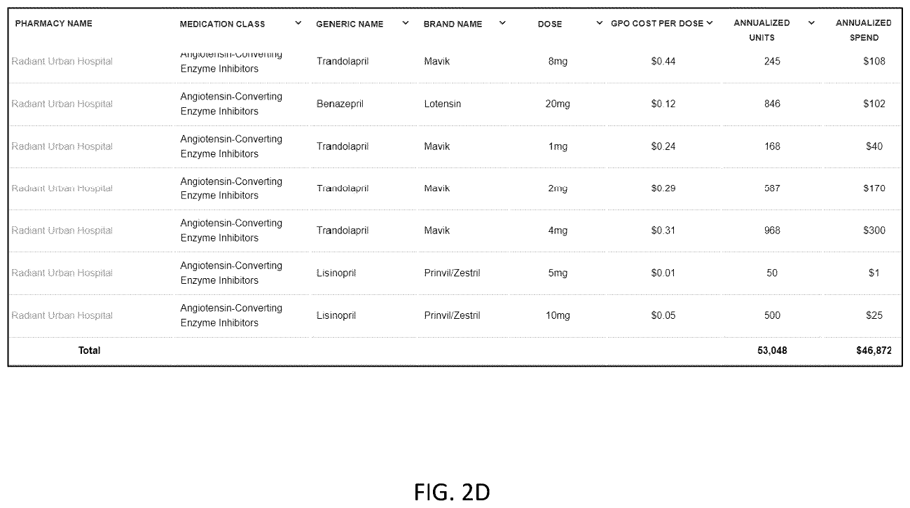

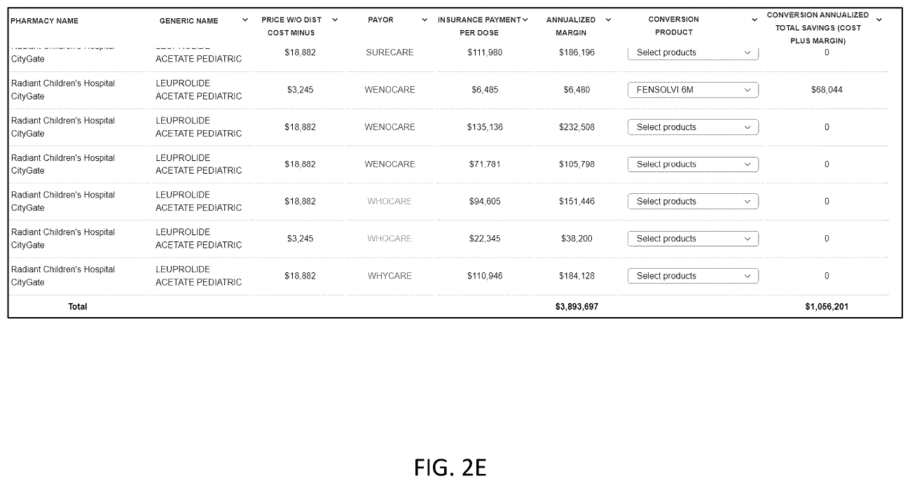

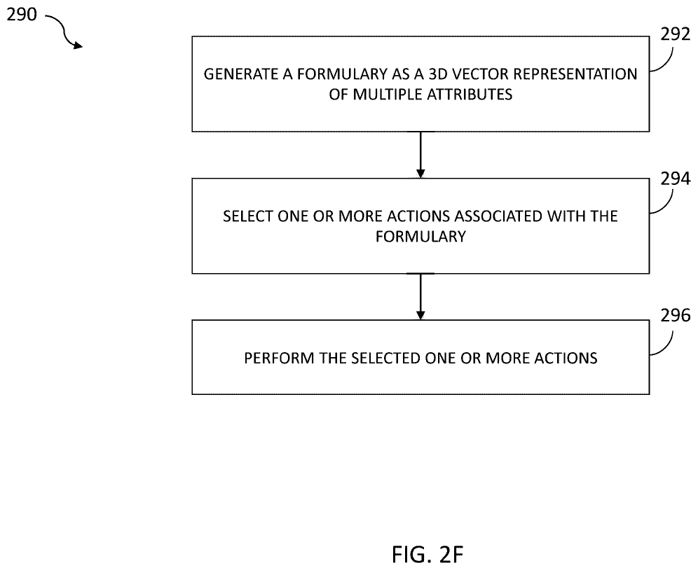

FIG. 2A is a block diagram illustrating example modules of the formulary system 150. The modules and/or components of the system 150 can be implemented with a combination of software (e.g., executable instructions, or computer code) and hardware (e.g., at least a memory and processor). Accordingly, as used herein, in some example embodiments, a component/module is a processor-implemented component/module and represents a computing device having a processor that is at least temporarily configured and/or programmed by executable instructions stored in memory to perform one or more of the functions that are described herein. The formulary system 150 includes a vector representation module 210, an action selection module 220, an action module 230, and a feedback module 240. In some embodiments, the vector representation module 210 is configured and/or programmed to generate a formulary as a three-dimensional vector representation of various attributes of a drug and related prescription or actions. The vector representation module 210 can be accessed by various modules or components (the dashboard 140) when searching for and obtaining information. In some cases, the module 210 can include information that links or maps multiple formularies to one another.

An example vector representation (of C-A-T) is as follows:
[0.0074, 0.0030, −0.0105, 0.0742, 0.0765, −0.0011, 0.0265, 0.0106, 0.0191, 0.0038, −0.0468, −0.0212, 0.0091, 0.0030, −0.0563, −0.0396, −0.0998, −0.0796, . . . , 0.0002]

In some embodiments, the action selection module 220 is configured and/or programmed to select one or more actions to perform with respect to a formulary. For example, the action selection module 220 can control or identify the actions to be performed, such as to render certain information, transmit or provide results of queries, search for information, request additional information, and so on.

In some embodiments, the action module 230 is configured and/or programmed to perform one or more actions associated with a formulary and/or in response to a query received via the platform 120. For example, the action module 230 causes the dashboard 140 to represent certain information via one or more dashboard elements.

In some embodiments, the feedback module 240 is configured and/or programmed to collect or obtain feedback (e.g., human feedback, such as scoring or other metrics), and provide the feedback to the training system 160. The action module 230 may then modify or update vector representations of formularies based on information obtained from the training system 160 (e.g., information output by one or more ML models using the feedback received from the feedback module 240).

As depicted, the formulary system 150 may interact with and/or be associated with the evaluation system 155 and other systems, such as a trials system 250 and/or a device system 260. The evaluation system 155 can include a vector representation 212 of data input into the system, such as a vector representation of various fields of data and/or parameters comparisons. Each medication and/or comparison of medications may thus be represented by a multi-dimensional vector that depicts a savings or other outcome of interchanging medications.

The system 155 may also include other modules, such as modules that receive or access data from the formulary system 150, the trials system 250 (e.g., clinical trial data), the device system 260 (e.g., medical device data), and so on. The system 155 may also prepare recommendations and/or perform actions to facilitate the interchange and/or surfacing of determined outcomes, such as outcomes that identify medications to utilize in replace of other medications.

FIG. 2B is a diagram illustrating various components of the evaluation system 155. An AI Engine 272, such as those described herein, may receive health system utilization data 270, which represents the use/utilization of certain medications in various clinical and/or medical applications. The AI Engine 272 performs validation 280 of the data and caches results 282 of the validation.

A review dashboard 274, which includes a user interface 275 accessible by various users (e.g., clinicians, hospital administrators, pharmacists, patients, caregivers, insurance administrators, analysts, government officials, pharmaceutical and medical device manufacturers, researchers, and so on), receives the results from the AI Engine 272, such as results of analyses comparing different medications and the interchangeability of the medications.

The review dashboard 274, as described herein, can present certain results as various graphical elements, to facilitate the comparison of medications and/or the selection of medications recommended for selection in place of other medications. For example, the evaluation system 155 may access data from multiple, disparate data sources, apply a machine-learning (ML) transformer to the multiple, disparate data sources to identify at least one pair of interchangeable medications, and perform an action based on the identification of the at least one pair of interchangeable medications. In some cases, the multiple, disparate data sources include data sources associated with clinical trials and multiple hospital systems and the ML transformer generates a prediction of multiple characteristics shared by the at least one pair of interchangeable medications.

FIGS. 2C-2E are diagrams illustrating example user interfaces presented by the review dashboard 274.

In some cases, the platform 120 may provide secure access to different categories of users according to their credentials and permissions. The platform 120 may record access instances and monitors their outcomes. In some embodiments, the platform 120 uses ML techniques to identify best decisions by users and recommend best practices.

A review module 285 facilitates review of the output of the AI Engine 272 and/or the data presented by the dashboard 274. Once reviewed (such as by a researcher or other expert), results 287 are confirmed and displayed via the dashboard 274 and/or provided to other systems or applications. The dashboard 274 may also provide data to a reinforcement learning module 276, which performs operations to optimize the various AI/ML processes utilized by the evaluation system 155 and described herein.

In some embodiments, the system 155 analyzes patient adherence and engagement data, recommending therapy routines (drugs and procedures) to improve overall health outcomes.

In some embodiments, the dashboard provides an interactive environment for the user to select actionable scenarios, adhere to their execution and evaluate results.

In some embodiments, the dashboard alerts the user to external changes in 3rd party data streams, e.g., insurance, clinical alternatives, best practices, scenario introduction, modification, and obsolescence.

In some embodiments, the dashboard provides regular feedback on ongoing clinical trials, with indications of drug/therapy performance above/below predefined clinical outcome parameter range.

Thus, in various embodiments, the platform 120 may perform methods or processes associated with a drug, medicine, and/or an associated/representative formulary. FIG. 2F is a flow diagram illustrating a method 290 for perform an action associated with a formulary. The method 290 may be performed by the platform 120 and, accordingly, is described herein merely by way of reference thereto. It will be appreciated that the method 290 may be performed on any suitable hardware.

In operation 292, the platform 120 generates a formulary as a formulary as a three-dimensional vector representation of multiple attributes. For example, the vector representation module 210 may generate a formulary that is a 3D vector of attributes for instructions and treatments related with a list of medicines or drugs represented by the formulary.

In operation 294, the platform 120 selects one or more actions associated with the formulary. For example, the action selection module 220 identifies the selected one or more actions via a data structure relating actions to formularies.

In operation 296, the platform 120 performs the selected one or more actions. For example, the action module 230 may cause an online dashboard to render and present information associated with the generated formulary and/or may generate a study snapshot that includes a background section, a literature review section, and a references section. In some cases, the action module 230 may perform some or all of the actions described herein.

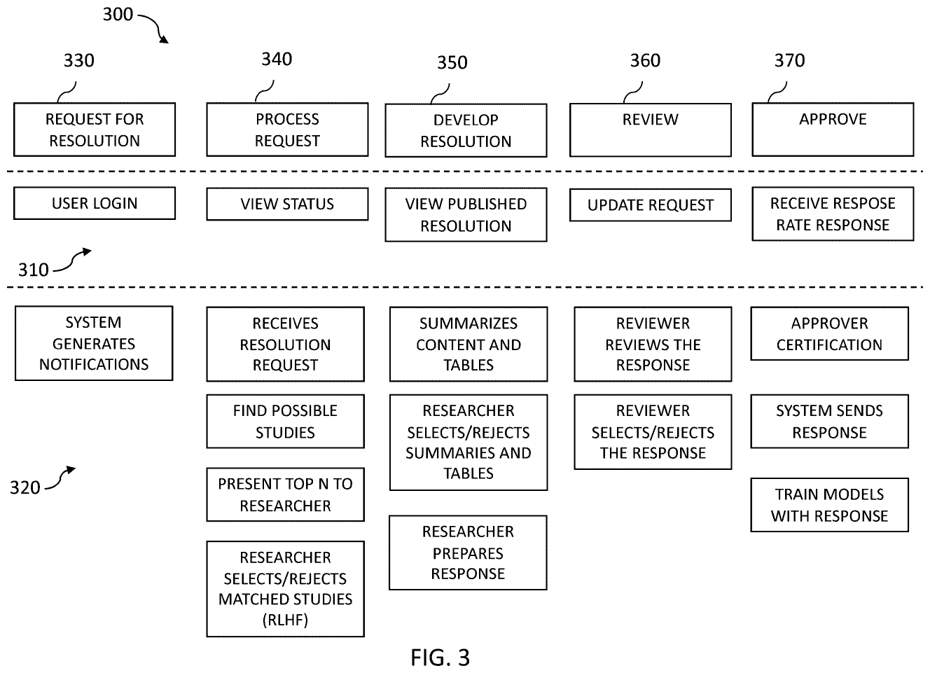

FIG. 3 is a diagram illustrating data flows 300 between components of the information exchange platform 120. The flow of data or information may be between user actions 310 and the ML or AI model 320, and include multiple operations, including:
A request submission operation 330 initiated by a user providing input (e.g., a request or query) to the platform 120 and the platform 120 generating one or more notifications;
A request processing operation 340 where the platform 120, via an AI engine or various ML models, selects multiple studies from various databases (e.g., a data bank) that are responsive to the request, and a human (e.g., researcher), provides input (e.g., accepts or rejects the studies) into an RLHF model;
A request resolution operation 350 where the platform 120 utilizes the AI engine to generate a summary of responses and uses the RLHF model to prepare a response to the request;
A review operation 360 where the platform 120 utilizes human feedback to review the prepared response, and/or an approval operation 370, where the platform 120 provides a response to the query and/or updates the training system 160 with the request/response to further train the models utilized by the platform 120; and so on.

### Examples of Identifying Drug Shortages Using the Platform

As described herein, the platform 120, in various embodiments, can facilitate the creation of a dashboard or other interface that identifies drugs or medicines that may be scarce or relatively unobtainable for a patient, doctor, hospital, pharmacy, or other inquiring entity.

For example, the scarcity of drugs may cause a shortage, leaving providers and pharmacies unable to procure them for patients. Hospitals face significant challenges in managing these shortages and finding timely alternatives. To address this issue, the platform 120 can support and provide a dashboard that identifies drugs at risk of scarcity. The dashboard may assign or determine a shortage risk score (or other metrics) to drugs listed in a formulary, providing a clear display of their respective scores.

For example, a higher shortage risk score indicates a higher likelihood of the drug going on shortage. The platform 120 may determine a shortage risk score based on several factors, such as: drug age, brand/generic availability, dosage forms-route of administration, dosage forms-modified release preparations, therapeutic category, pricing, drug recalled, number of manufacturers, location of manufacturer, disaster in geographic location of manufacturer, manufacturing pause or discontinuation, number of API suppliers, disaster in geographic location of API supplier information, and so on.

In some cases, the shortage risk score may be the sum of points assigned to individual factors or may be based on more complex or weighted determinations. In some cases, the dashboard may display drugs with the highest shortage risk score at a top of a list, enabling users to determine a plan before the drug is unavailable, among other benefits.

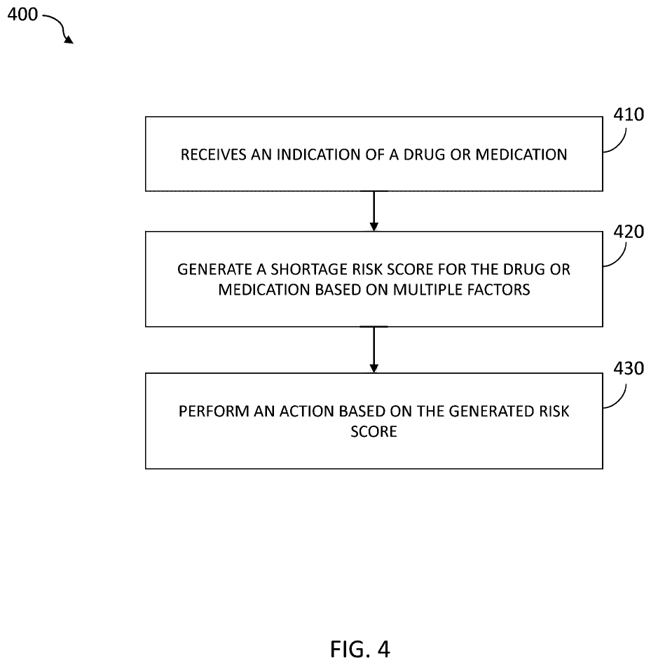

FIG. 4 is a flow diagram illustrating an example method 400 for determining drug shortages using the platform 120. The method 400 may be performed by the platform 120 and, accordingly, is described herein merely by way of reference thereto. It will be appreciated that the method 400 may be performed on any suitable hardware. The method 400 presents an overall schema for scoring drug availability risk, based on the factors described herein.

In operation 410, the platform 120 receives an indication of a drug or medication. For example, the platform 120 may receive a search query or other query, as described herein.

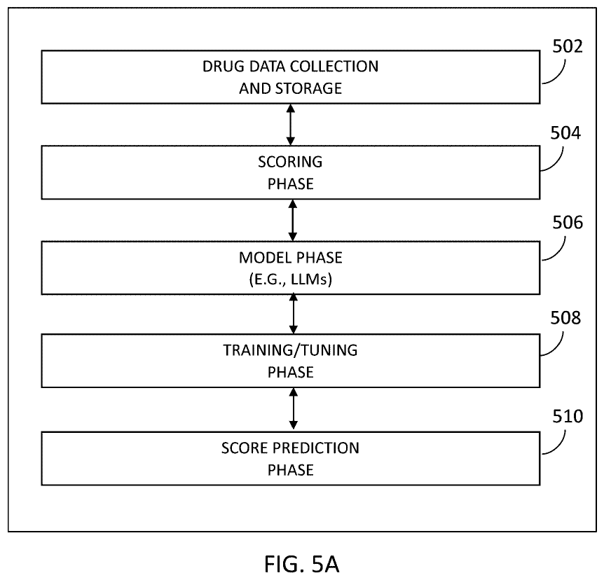

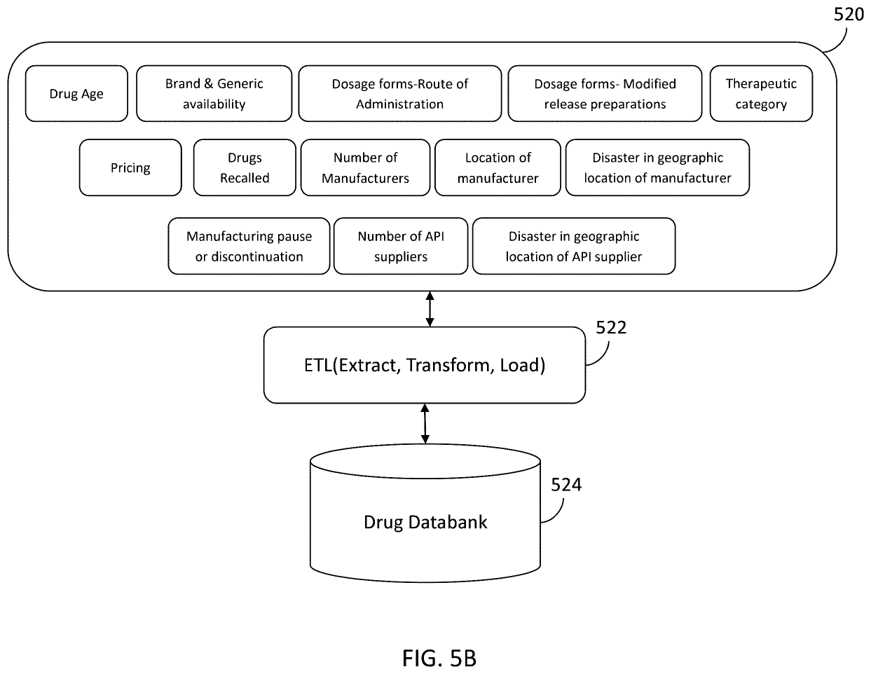

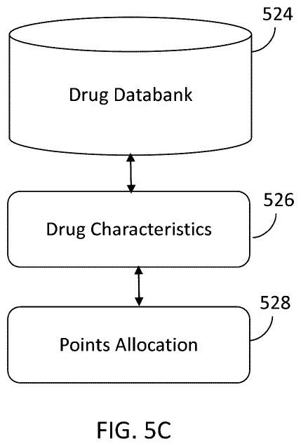

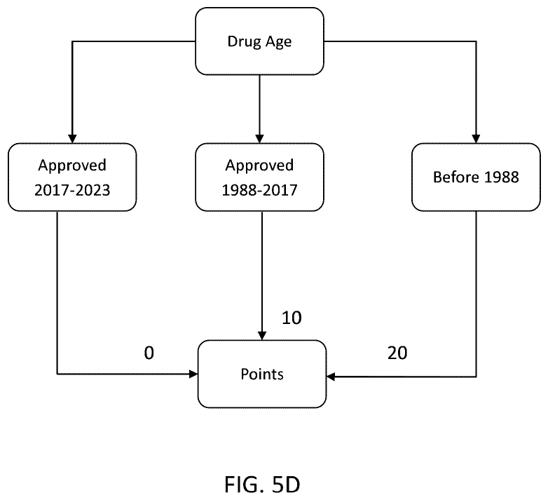

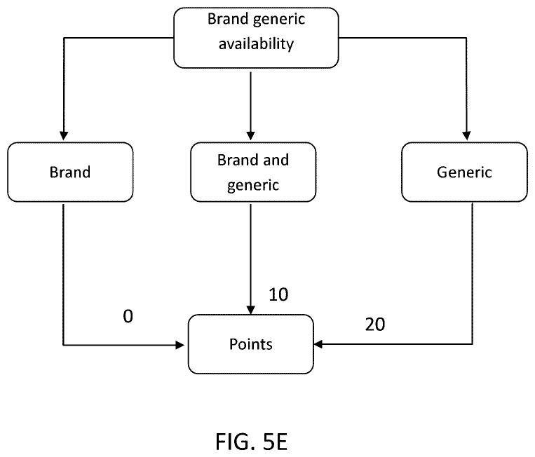

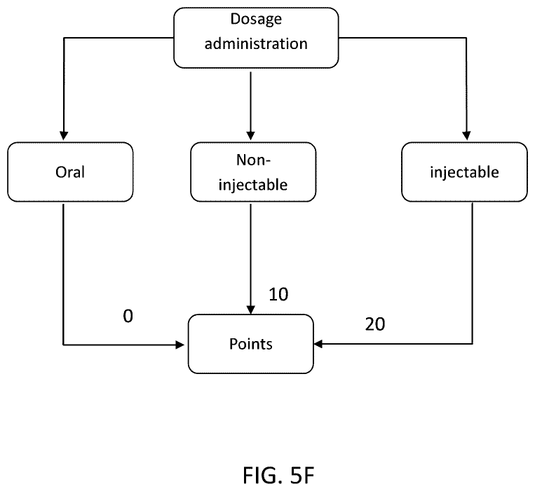

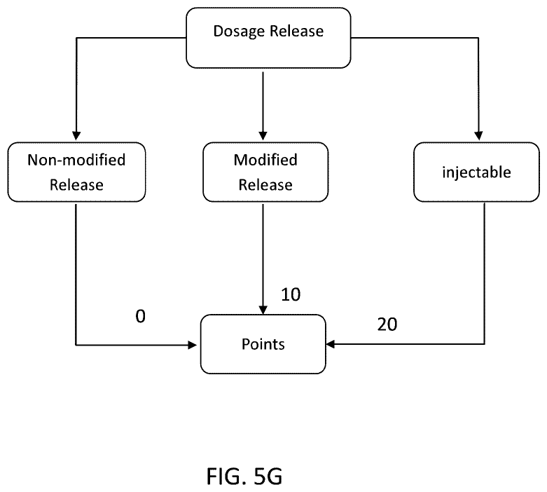

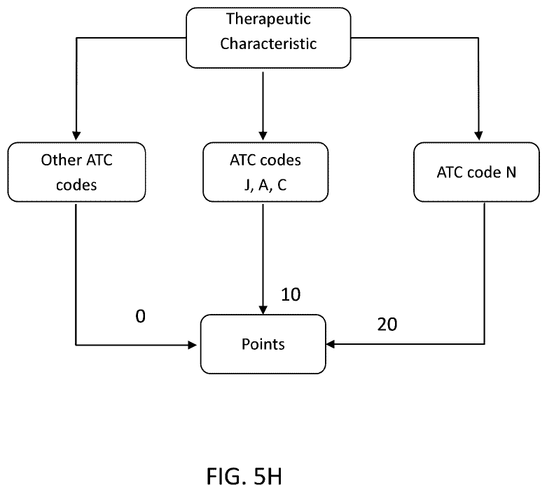

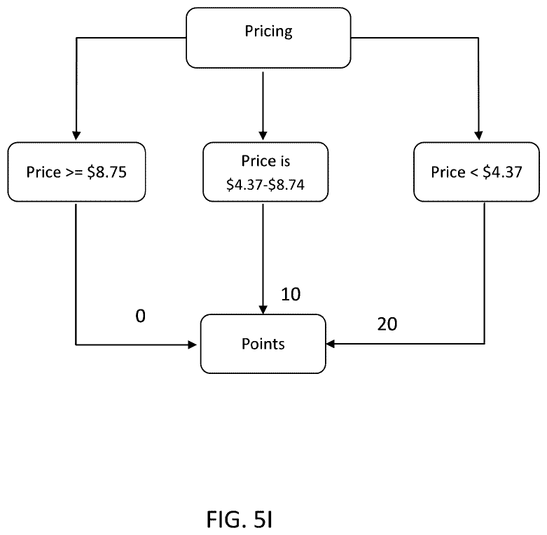

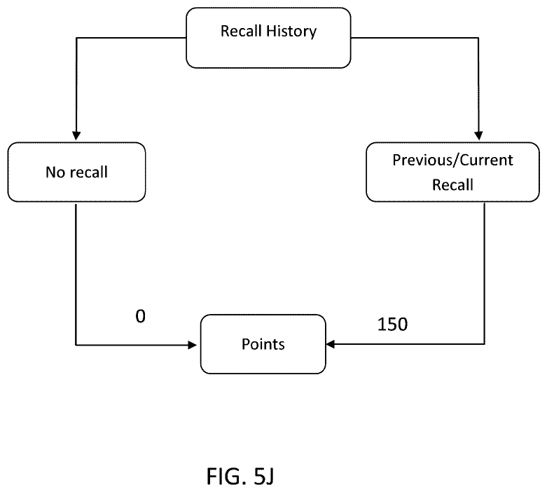

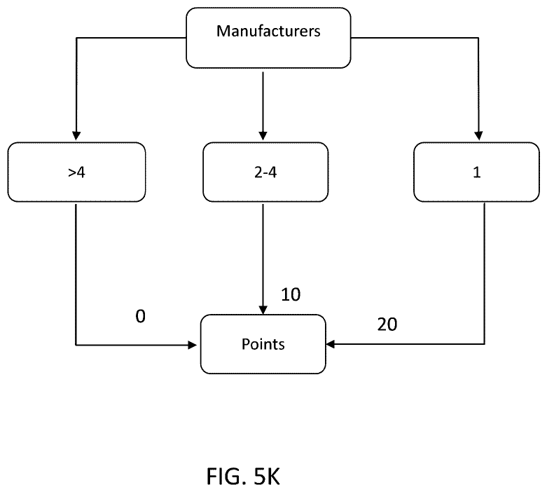

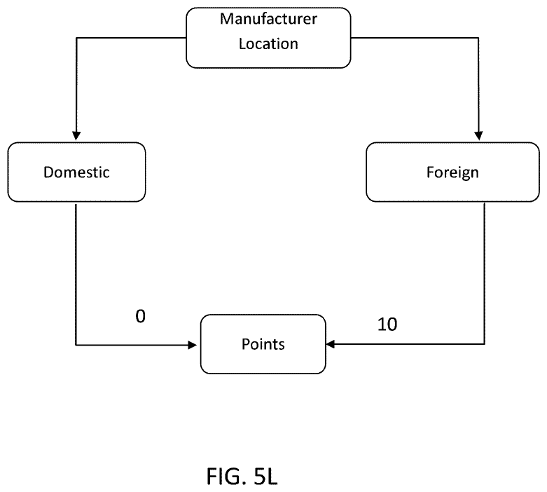

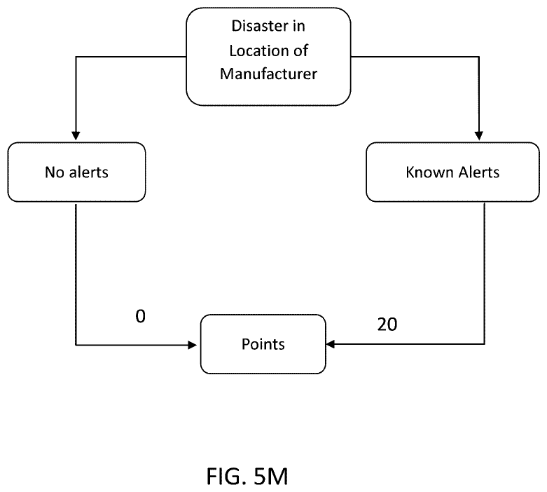

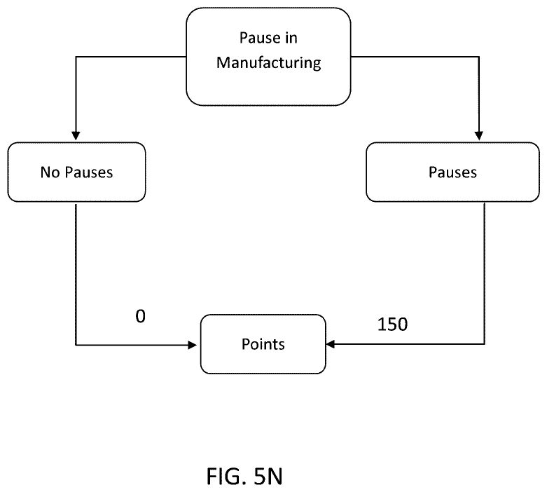

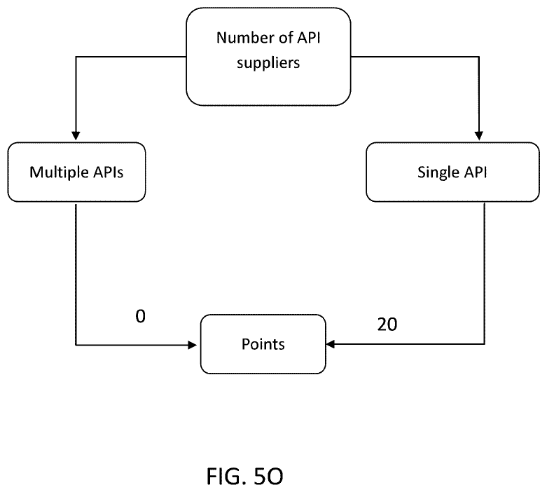

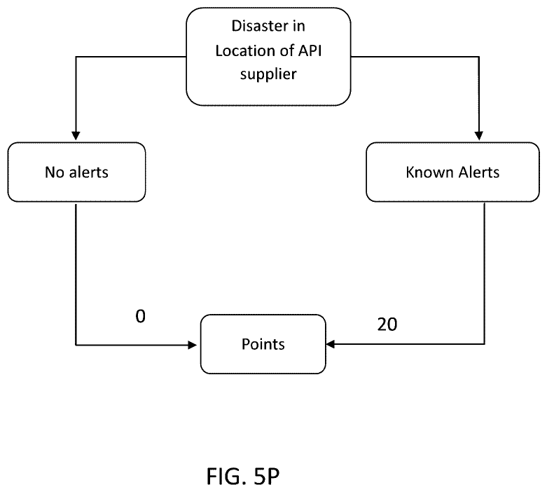

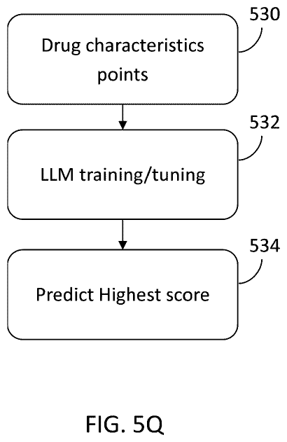

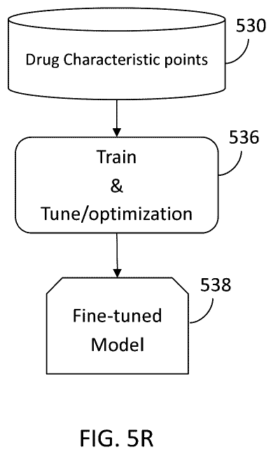

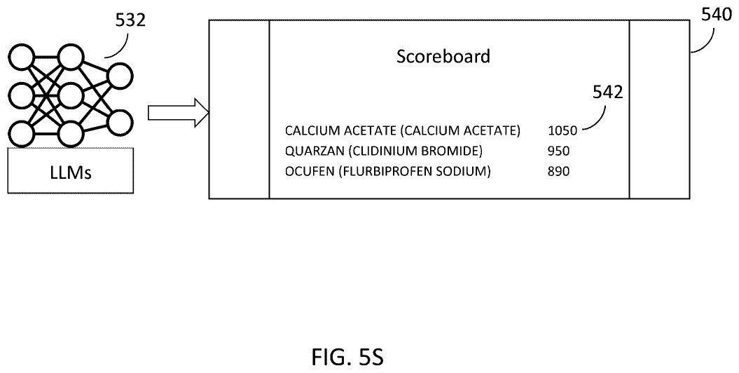

In operation 420, the platform 120 generates a shortage risk score for the drug or medication based on multiple factors, and, in operation 430, performs an action based on the generated shortage risk score. For example, the platform 120 may input the multiple factors into an LLM (as described herein and generate a prediction for the shortage risk score. FIGS. 5A-5S provide additional details regarding the scoring of drugs (e.g., shortage risk scores) in view of their availability or risk factors.

In some cases, the platform 120 integrates data from multiple sources, such as FDA reports, third party publications, patient charts, utilization information, billing data, payor requests/responses, pharmacy requests for change, wellness trends, epidemiological data, drug manufacturing locations, drug pricing data, drug cost data, and so on.

FIG. 5A depicts various phases that may be performed by the platform 120, including a drug data collection and storage phase 502, a scoring phase (e.g., awarding points based on drug characteristics) 504, a model phase (e.g., use of LLMs) 506, a training and fine-tuning phase 508, and a score prediction phase 510.

During the drug data collection and storage phase 502 (see FIG. 5B), the platform 120 accesses, collects, or obtains data 520, such as data associated with drug age, brand/generic availability, dosage forms-route of administration, dosage forms-modified release preparations, therapeutic category, pricing, drug recalled, number of manufacturers, location of manufacturer, disaster in geographic location of manufacturer, manufacturing pause or discontinuation, number of API (Active Pharmaceutical Ingredient) suppliers, disaster in geographic location of API supplier information of the drug, and so on. The platform 120 may perform various extract, transform load (ETL) processes 522 for the collected information, and stored the information a database (e.g., a Drug Databank 524).

During the scoring phase 504 (see FIG. 5C), the platform 120 assigns/allocates 528 points to some or all drug characteristics 526 of drugs in the Drug Databank 524. For example, the platform 120 may assign the brand/generic availability characteristic 0 points for a brand, 20 points for a generic, and 10 points for both brand and generic.

Thus, the platform 120 may allocate 528 points for different characteristics by following a rules-based process (e.g., an if/then process, see FIG. 4). Details regarding example scoring for some of the possible characteristics will now be described.

FIG. 5D depicts the allocation of points for a “drug age” characteristic. The points allocated for the drug age characteristic are based on an approval year period, where 0 points are allocated for period 2017-2023, 10 points are allocated for period 1988-2017, and 20 points are allocated for drugs approved before 1988.

FIG. 5E depicts the allocation of points for a “brand/generic” characteristic. The points allocated for the brand/generic availability characteristic are based on the brand and can include assigning 0 points for a brand, 10 points for both a brand and a generic, and 20 points for a generic.

FIG. 5F depicts the allocation of points for a “dosage administration” characteristic. The points allocated for the dosage forms-route of Administration characteristic are based on the route of administration. A drug is assigned 0 points for oral, 10 points for non-injectable and non-oral, and 20 points for injectable.

FIG. 5G depicts the allocation of points for a “dosage release” characteristic. The points allocated for the dosage forms-modified release preparations characteristic are based on the release preparations. A drug receives 0 points for non-modified release preparations, 10 points for modified release preparations, and 20 points for injectable drugs. Modified-release preparations may be defined as Controlled Delivery (CD), Controlled Release (CR), Continuous control (CC), Delayed release (DR), Extended release (ER), Long acting (LA), Long acting release (LAR), Modified release (MR), Prolonged release (PR), Sustained Action (SA), Sustained release (SR), Timed release (TR), Extra Long (XL), Extended release (XR), and Extended time (XT).

FIG. 5H depicts the allocation of points for a “therapeutic” characteristic. The points allocated for the Therapeutic category characteristic are based on ATC (Anatomical Therapeutic Chemical) codes for the drugs. A drug receives 0 points for all other ATC codes, 10 points for ATC code J, C and A, and 20 points for ATC code N.

FIG. 5I depicts the allocation of points for a “pricing” characteristic. The points allocated for the pricing characteristic are based on the unit price for the drug. A drug receives 0 points for its unit price being >=$8.75, 10 points for its unit price being $4.37-$8.74, and 20 points its unit price being $0-$4.46.

FIG. 5J depicts the allocation of points for a “recall” characteristic. The points allocated for the drug recalled characteristic are based on a previous or current recall of the drug. The drug receives 0 points for no previous or current recall and 150 points for a current recall of the drug.

FIG. 5K depicts the allocation of points for a “manufacturer” characteristic. The points allocated for the manufacturer characteristic are based on the manufacturer's number or identity. A drug receives 0 points for a number that is >4, 10 points for a number between 2-4, and 20 points for the number 1.

FIG. 5L depicts the allocation of points for a “manufacturer location” characteristic. The points allocated for the manufacturer location characteristic are based on a geographical location of the drug manufacturer. A drug receives 0 points for a US based (or domestic) manufacturer, and 10 points for a manufacturer located in a foreign country (from the country of the requester).

FIG. 5M depicts the allocation of points for a “location disaster” characteristic. The points allocated for this characteristic are based on the geographic location. A drug receives 0 points for no alerts at the location and 20 points for an alert at the location.

FIG. 5N depicts the allocation of points for a “manufacturer pause” characteristic. The points allocated for this characteristic are based on the manufacturing status of the drug (e.g., whether the drug has been paused or discontinued). The drug receives 0 points for an active status and 150 points for a paused or discontinued status.

FIG. 5O depicts the allocation of points for a “supplier API” characteristic. The points allocated for this characteristic are based on the supplier API number. A drug receives 0 points for an API number >1 (e.g., multiple suppliers) and 20 points for a single supplier.

FIG. 5P depicts the allocation of points for a “supplier location” characteristic. The points allocated for this characteristic are based on the geographic location of API suppliers. A drug receives 0 points for no alerts at the supplier location and 20 points for an alert at the supplier location.

During the model phase 506 (see FIG. 5Q), one or more large language models (LLMs) 532 or other models may consider or use different drug characteristic points or scores 530 as input, process the input, and generate a prediction 534 of a shortage risk score for each drug (including a highest risk score or group of scores). As described herein, the shortage risk score may represent or be based on a determined/predicted scarcity for the drug or drugs.

During the training phase 508 (see FIG. 5R), the LLMs are trained/tuned 536 using the assigned drug characteristic points 530 (e.g., their performance may improve over time and training), generating and/or creating a fine-tuned model 538.

FIG. 5S depicts the score prediction phase 510, presenting a scoreboard 540 (or dashboard or other UI) of drugs with the highest predicted risk scores 542 (e.g., predictions of potential drug shortages or scarcities in real-time). Via the dashboard, the platform 120 may act as a decision support tool for healthcare providers, manufacturers, and policymakers to proactively address drug shortages via the presented information.

Thus, the platform 120, in some embodiments, can utilize obtained information and ML models to predict or determine drugs or medicines that may be scarce or unavailable in a future time period.

In some embodiments, the platform 120 recommends alternative drugs or therapies that are based on available during target periods. In some embodiments, the user (e.g., a doctor) pre-authorizes alternatives for release via the platform 120. In some embodiments, the platform 120 requests feedback on outcomes of the use alternatives.

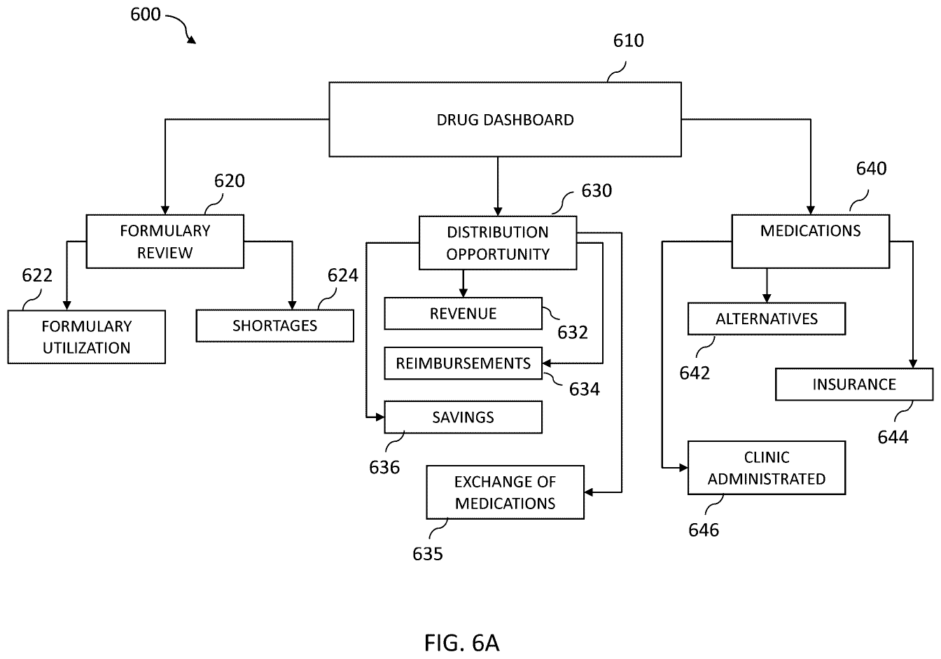

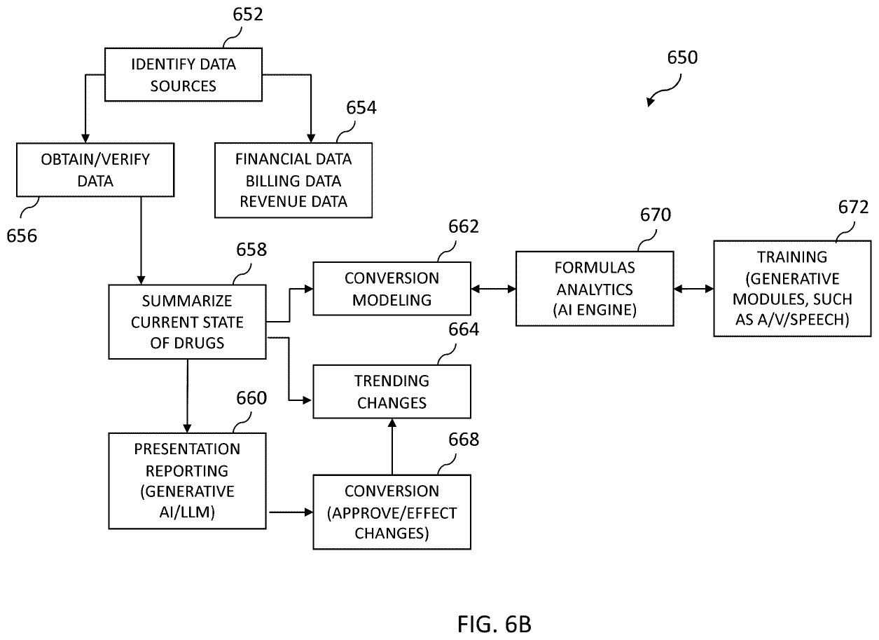

In some embodiments, the dashboard 140 can be associated with the formulary system 150. FIG. 6A is a diagram 600 illustrating a dashboard 610 provided by the platform 120. As depicted, the dashboard 610 can facilitate a formulary review 620, identify financial or distribution opportunities 630 (e.g., via medication exchanges 635, see FIG. 6B), can identify medications 640, and so on.

For example, the formulary review 620 may identify formulary utilization 622 and/or shortages 624 information. Further, the identification of financial or distribution opportunities may be associated with revenue 632 enhancements or actions, reimbursements 634 enhancements or actions (e.g., Medicare), savings 636, and/or the medication exchanges 636. Also, the identification of medications can include the identification of alternatives 642 and/or be associated with insurance-based actions 644 or clinic actions 646.

FIG. 6B presents various information flows 650 during the medication exchange operations described herein (e.g., the medication exchanges 635). First, the platform 120 may identify data sources 652, such as various data stores 654 (e.g., financial data stores, billing data stores, revenue data stores, and so on). The platform 120 obtains/verifies the data 656 and determines a summary of a current state of a drug or drugs 658. For example, the summary may include a current volume, cost, expenses (e.g., annualized by site), revenue, utilization, and so on. The platform 120 may utilize an LLM or generative AI models to generate, determine, and/or perform presentation reporting 660 for the current state of the drug or drugs.

Further, the platform 120 may perform conversion modeling 662 (e.g., identifying opportunities for changing/exchanging drugs/medications) based on the current state of the drug or drugs, utilizing formulas and/or analytics 670, such as those performed by the AI engines described herein (e.g., and trained using various generative models 672). The platform 120 may also identify, determine, and/or predict trending changes 664 to the current state of the drug or drugs, such as by determining conversions 668 based on the presentation reporting 660.

In some embodiments the dashboard 140 presents best practices viewed across different healthcare systems (e.g., via a heatmap) with dimensions such as costs per drug class, therapy class, patient response to drug, efficacy, dosing, and so on. In some cases, the dashboard 140 provides personalized views into the formulary at different levels: drug, drug class, patient, doctor, hospital, healthcare system, region, and so on.

In some embodiments, the dashboard 140 presents hypotheses for drug substitution (e.g., formulary updates), based on efficacy, costs, side effects, shortages, and so on. After the user chooses a hypothesis for further exploration, the dashboard 140 presents recommendations on target medical conditions, patient body, control groups, clinical environment, and so on. The dashboard 140 may also coordinate interactions between multiple users in order to evaluate the hypothesis. Once a hypothesis is accepted for clinical implementation, the dashboard 140 enables the user to designate participants and feedback collection mechanisms.

Upon receiving feedback within a predetermined period of time, the platform is enabled to establish whether the hypothesis is confirmed by the clinical implementation. Furthermore, the dashboard is enabled to accept approval of the implementation and formulary update based on the results.

Thus, in some cases, the dashboard can display or present current medications on formulary as well as opportunities for therapeutic interchanges to optimize it, promoting cost effective care in a scalable and readily available manner, among other benefits.

### Examples of Information Exchange Using the Platform

As described herein, in some embodiments, the platform 120 utilizes RLHF or other reinforcement learning models to adapt its operations based on user preferences, search patterns, corrective actions, and so on. The platform 120, therefore, combines machine learning with human expertise and intervention to update and enhance its performance, which may be limited when based on one or the other.

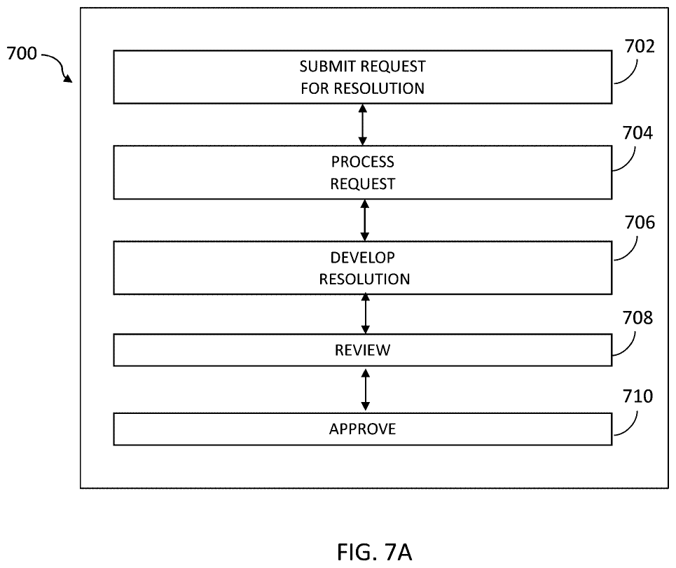

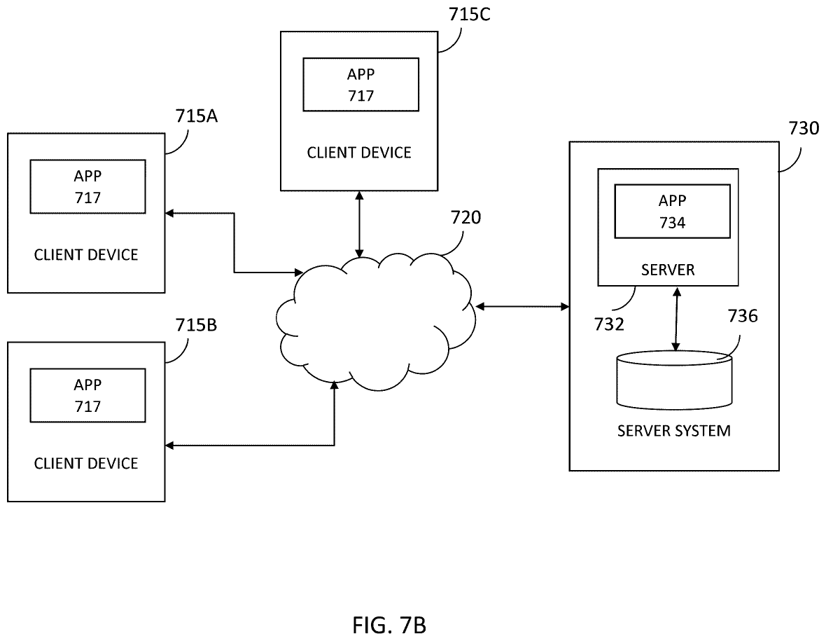

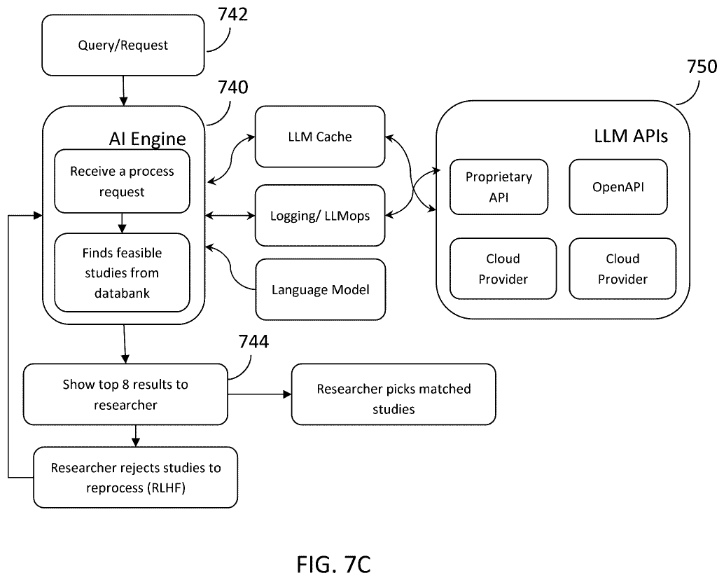

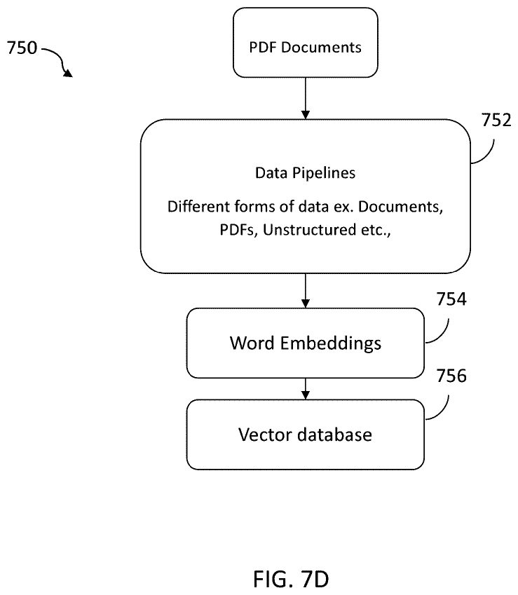

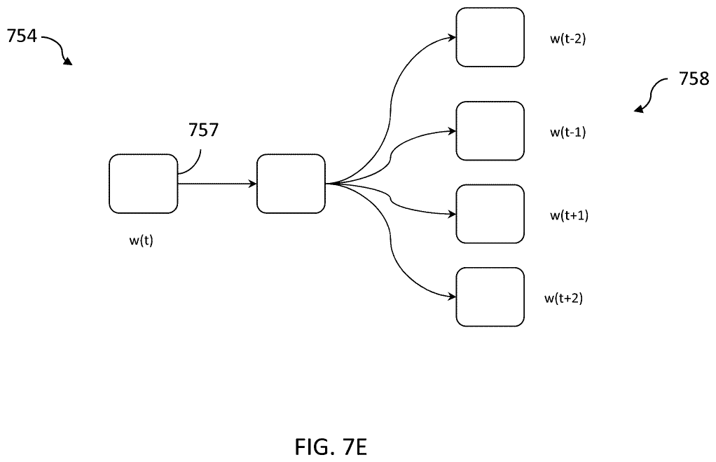

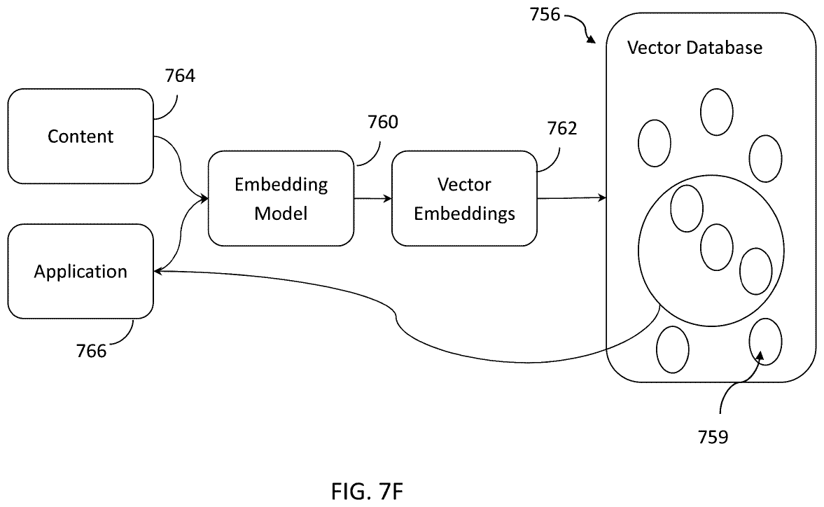

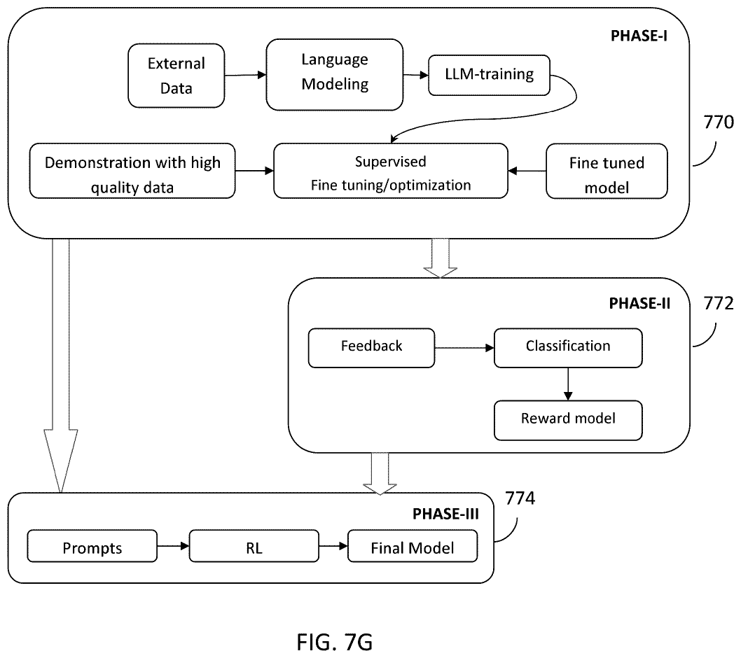

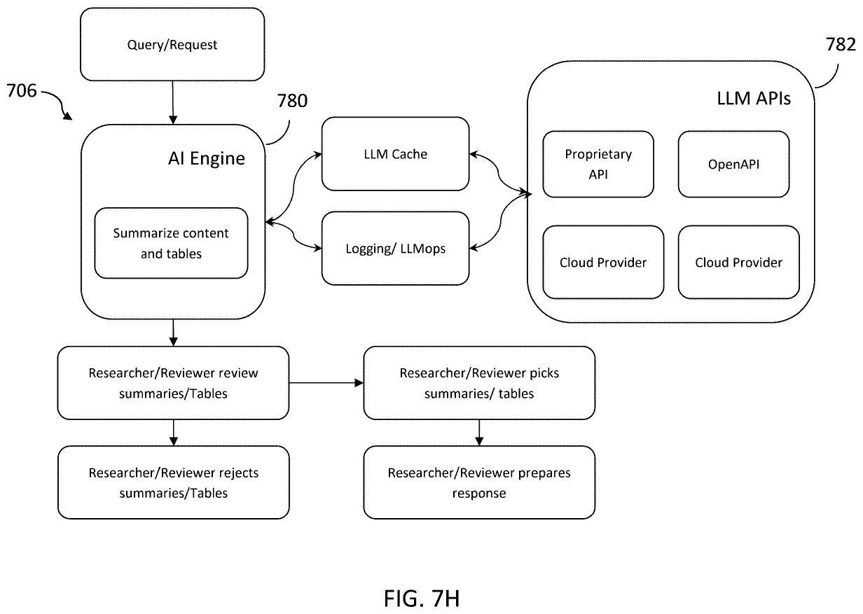

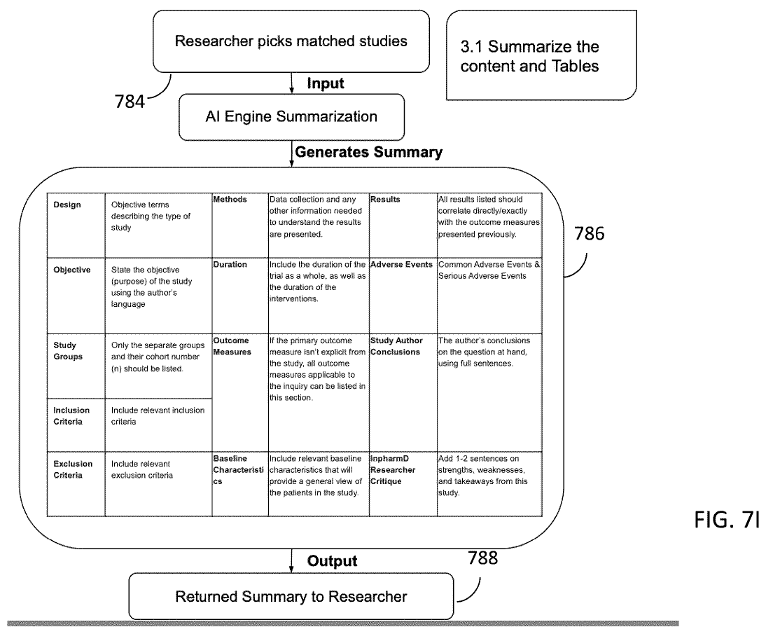

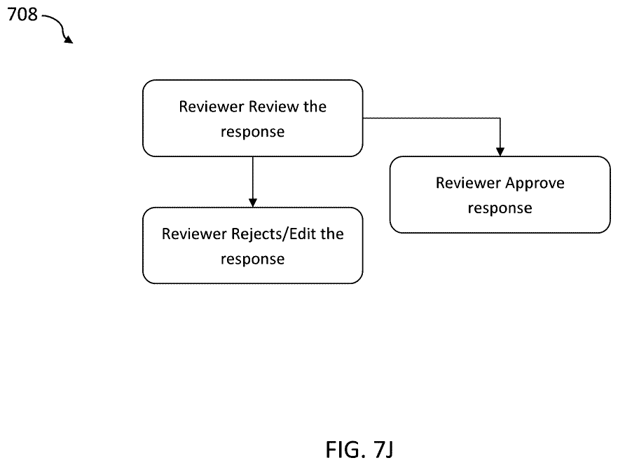

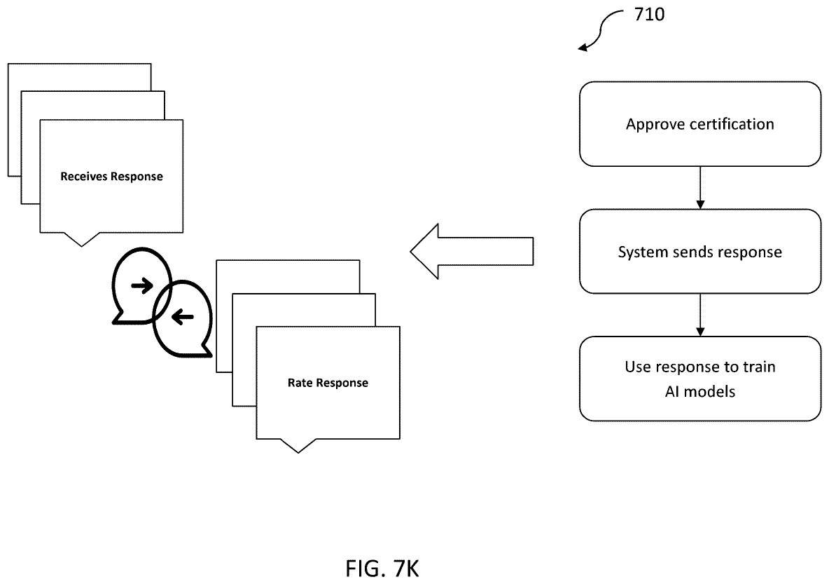

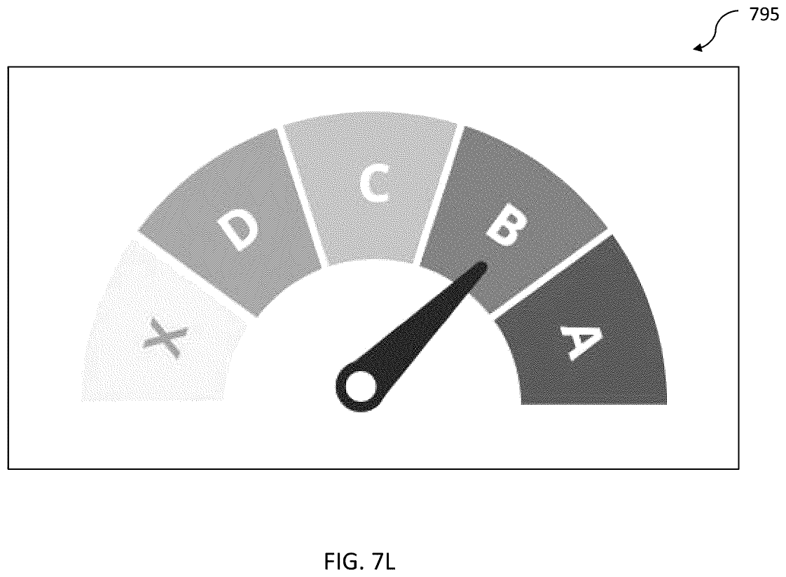

FIG. 7A depicts a structure of an RHLF model 700. The RHLF model, or reward model, is trained to predict whether a given output is good (e.g., a high reward) or bad (e.g., a low reward). In some cases, the RLHF model may improve the robustness and exploration of RL agents or models, such as when reward functions are sparse or noisy. The RHLF model may include a submit request module 702, a process request module 704, a develop resolution module 706, a review module 708, and an approve module 710.

FIG. 7B depicts the submit request module 702, which may include an authentication function. The submit request module 720 may include client devices 715A-C that communication with a server system 730 over a network 720. For example, a provider associated with the client device 715A sends requests and attempts to obtain authentication from a server 732. Client applications 717 accept requests from three different users: an admin user (e.g., associated with the client device 715B), the provider, and a researcher (e.g., associated with the client device 715C). These requests are sent to an application 734 running on the server 732 through the network 720, which gets the appropriate information (e.g., retrieved via a database 736) and returns it back to the client devices 715A-C.

FIG. 7C depicts the process request module 704. The process request module 704 includes an AI engine 740 and language models (e.g., LLMs) 750. The AI engine 740 considers a query or request 742 from the provider, processing the request using language modeling operations, as described herein. For example, the module returns a top 8 results output 744 to a researcher. The researcher may verify the results returned by the AI engine; the results are considered if they match the query; otherwise, the researcher adds relevant knowledge to the results and returns them back to the AI engine 740. This process (e.g., RLHF) continues until the researcher is satisfied (and may certify the results).

The language model 750 (see FIG. 7D) considers the input in different formats 752, prepares word embeddings 754, and constructs a vector database 756. In some cases, the word embeddings 754 are a type of word representation that allows words with similar meaning to have a similar representation. The vector databases 756 are specialized storage systems designed for efficient management of dense vectors and support advanced similarity searches for drugs, medications, formularies, and so on.

FIG. 7E depicts word embeddings, where words and documents are represented in the form of numeric vectors 758, allowing similar words to have similar vector representations. The extracted features are fed into a machine learning model so as to work with text data and preserve the semantic and syntactic information. Once the information is received in its converted form, it is used by NLP algorithms that digest these learned representations and process textual information.

FIG. 7F depicts the vector database 756, which is a type of database that stores data (e.g., a formulary) as high-dimensional vectors, mathematical representations of features or attributes. Each vector has a certain number of dimensions 759, which can range from tens to thousands, depending on the complexity and granularity of the data.

For example, the platform 120 may use an embedding model 760 to create vector embeddings 762 for content 764 to be indexed. The vector embedding 762 is inserted into the vector database 756, with some reference to the original content the embedding was created from. When an application 766 issues a query, the platform 120 uses the same embedding model 760 to create embeddings 762 for the query and uses those embeddings to query the database 756 for similar vector embeddings. In some cases, these similar embeddings are associated with the original content that was used to create them.

In some embodiments, a RLHF process, depicted in FIG. 7G, may include phases, such as a training and tuning phase 770, a reward phase 772, and a reinforcement phase 774.

Via the training and tuning phase 770, external data is collected, and the platform 120 pretrains the language model and tunes the model. The platform, during the reward phase 772, guides the reinforcement learning agent during training using the reward model. The reward model encodes the preferences and guidance provided by humans into a form that the agent can understand and optimize. Finally, the RL model is prepared in the reinforcement phase 774.

FIG. 7H depicts the develop resolution module 706, which includes an AI engine 780, summary content, and language modeling 782. The AI engine 780 considers the matched studies that are picked by researchers (e.g., received from process request module depicted in FIG. 7C), processing the list of studies using the language models 782. The AI engine 780 prepares a summary of the content (including Design, Objective, Study Groups, Inclusion & Exclusion Criteria, Methods, Duration, Outcome Measures, Baseline Characteristics, Results, Adverse Events, Study Author Conclusions, and so on). The researcher reviews the summary content returned by the AI engine 780. If the summary matches studies picked by researchers, the AI engine 780 considers and prepares the response; otherwise, the researcher rejects the summary content returned by the AI engine 780.

FIG. 7I depicts an application of the platform 120, such as the creation and output of a comprehensive summary of research content and associated tables developed by the platform 120. The process starts with the researcher's input 784, which includes the matched studies that are picked by researchers. The input is then fed into an AI engine specialized in summarization. The AI engine analyzes the content and generates a summary 786 (e.g., part of a snapshot, as described herein) that encompasses various aspects described herein. This summarized content is systematically organized within a “Summary of Research Content” section. Finally, the summarized content, containing condensed and relevant information, is presented as an output (e.g., the snapshot) 788, which is then returned to the researcher. This process streamlines the presentation of essential research details, enabling the researcher to access a consolidated overview of the study's key elements.

The review phase 708, depicted in FIG. 7J, contains a response status. The response mainly contains custom response text, overview of clinical guidelines, overview of commentaries, consensus statements, and review articles, overview of meta-analyses, overview of other tertiary sources and references, and so on. The reviewer may review the response returned by the AI engine. If the response matches a query, it will be considered and approved; otherwise, the researcher will reject or request to edit the reviewed response.

The approval phase 710, performed by an approval module depicted in FIG. 7K, performs approval certification. The researcher/reviewer receives responses from AI engines and approves based on the background, relevant prescribing information and literature review. The approved response will be rated. This response will be used for training the AI models. For example, FIG. 7L presents a rating element 795 (e.g., displayed along with a summary or snapshot), where “A” is a high rating, “D” is a low rating, and “X” denotes a lack of relevance. Of course, other rating or scoring schema may be used. Upon approval, the platform 120 may embed authentication and verification data (e.g., such as via blockchain technology).

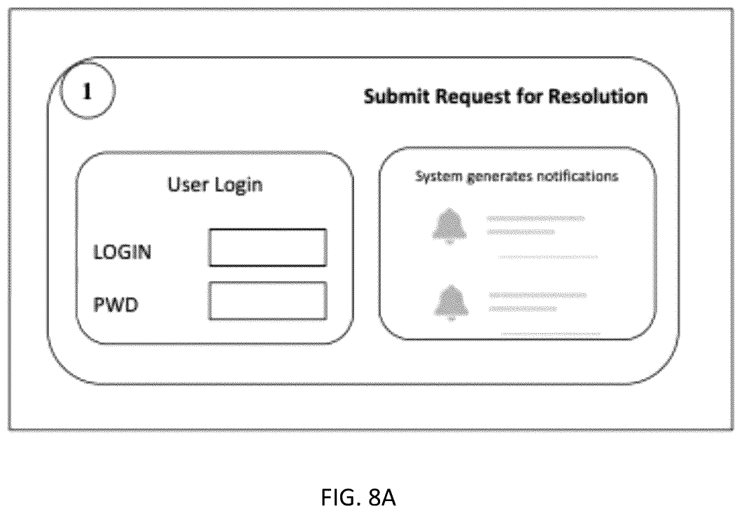

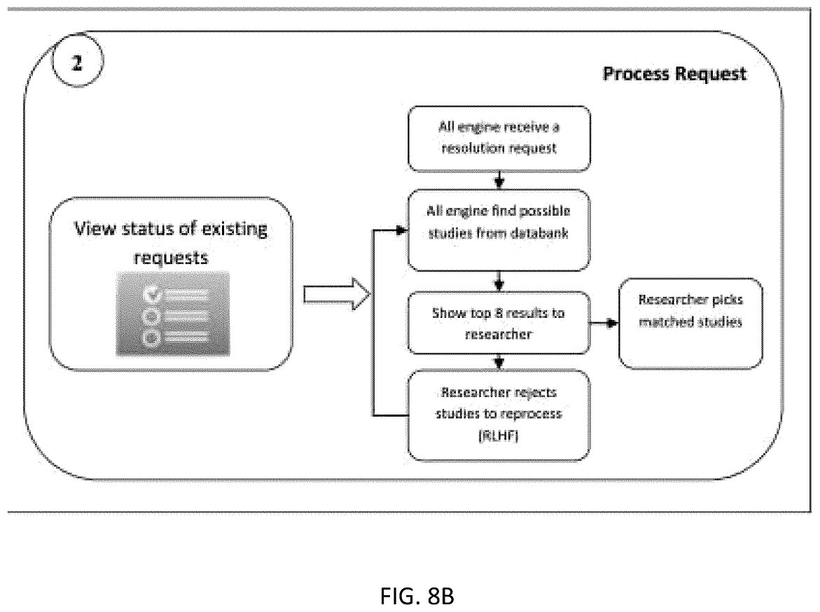

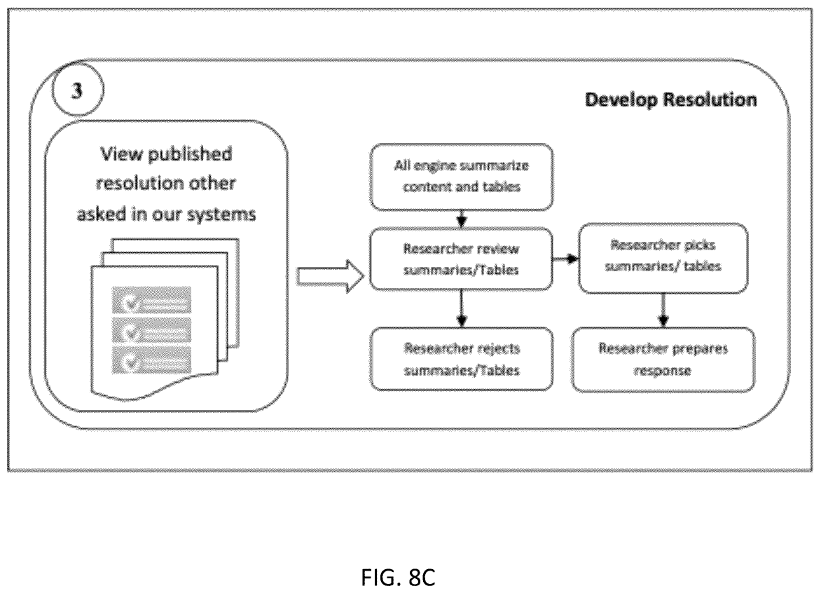

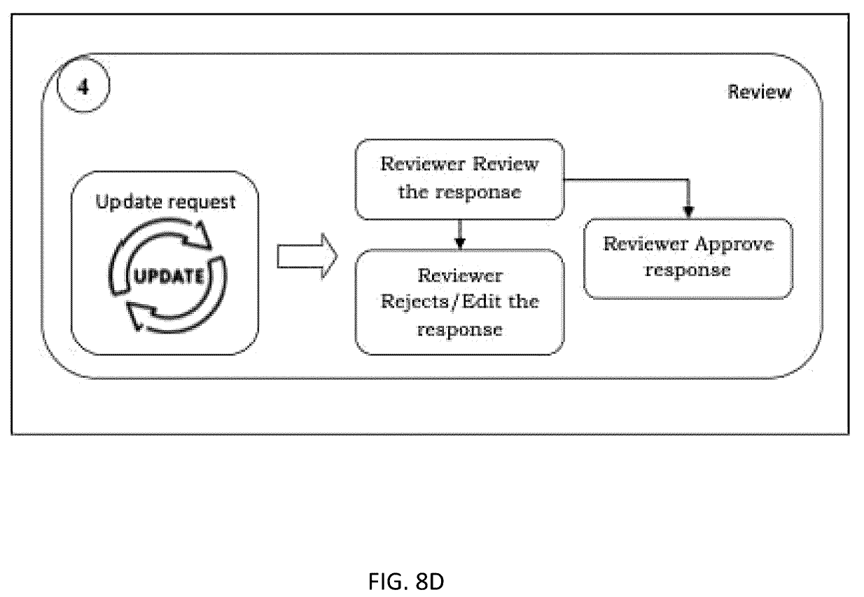

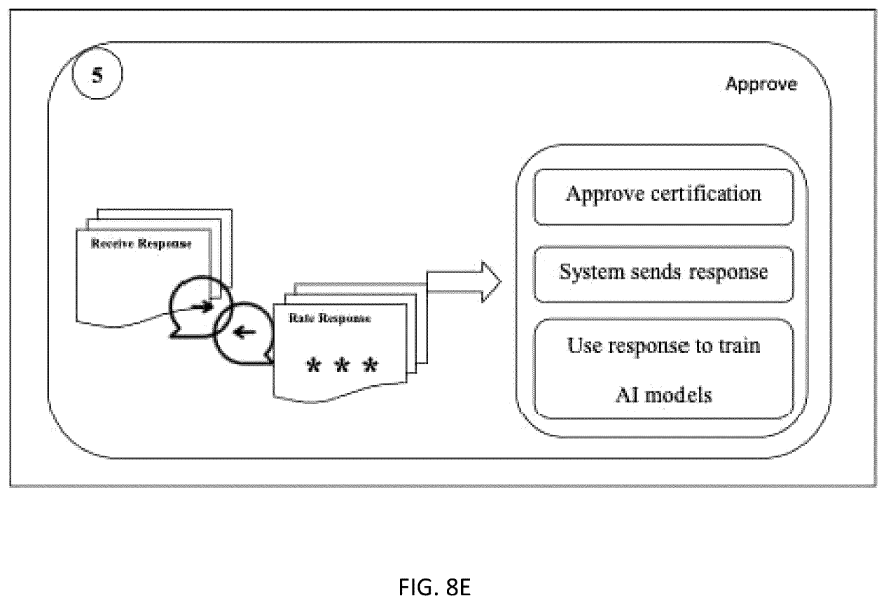

FIGS. 8A-8E are diagrams illustrating details of a user experience via the platform 120. As described herein, the platform 120 includes a structure that facilitates interactions between users, AI engines, backend processes, and various servers/devices. FIGS. 8A-8E depict example UIs presented by the platform 120, as well as their associated processes and user experiences.

For example, FIG. 8A depicts a UI that facilitates the submittal of requests from users, FIG. 8B depicts a UI associated with processing requests, FIG. 8C depicts a UI associated with resolving requests, FIG. 8D depicts a UI associated with reviewing generated responses, and FIG. 8E depicts a UI for approving and performing actions with responses.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the electric bike and bike frame may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

What is claimed is:

1. A system, comprising:
a vector representation module that is configured to generate a formulary as a three-dimensional vector representation of multiple attributes;
an action selection module that is configured to select one or more actions associated with the formulary; and
an action module that is configured to perform the selected one or more actions.

2. The system of claim 1, further comprising:
a feedback module that is configured to:
obtain feedback associated with the selected one or more actions;
send the obtained feedback to a machine learning based training system;
receive information from the machine learning based training system; and
cause the action module to modify the three-dimensional vector representation of the formulary.

3. The system of claim 2, wherein the information received from the machine learning based training system includes information generated by a reinforcement learning from human feedback (RLHF) model.

4. The system of claim 1, wherein the formulary comprises a list of medicines or drugs associated with a prescription, and wherein the three-dimensional vector representation of multiple attributes includes attributes associated with instructions and treatments related with the list of medicines or drugs.

5. The system of claim 1, wherein the formulary is an actionable data object that is related to other actionable data objects representing other formularies.

6. The system of claim 1, wherein the vector representation module generates the formulary as the three-dimensional vector representation of multiple attributes based on data collected from one or more unstructured data sources.

7. The system of claim 6, wherein the one or more unstructured data sources include medical papers, clinical information, study results, or drug information.

8. The system of claim 1, wherein the action selection module identifies the selected one or more actions via a data structure relating actions to formularies.

9. The system of claim 1, wherein the action module causes an online dashboard to render and present information associated with the generated formulary.

10. The system of claim 1, wherein the vector representation module generates the formulary as the three-dimensional vector representation based on receiving a search query that requests information about a drug or treatment.

11. The system of claim 1, wherein the action module generates a study snapshot that includes a background section, a literature review section, and a references section.

12. The system of claim 11, wherein the literature review section includes a scoring indicator that assigns a study quality metric to literature presented by the literature review section.

13. A non-transitory, computer-readable medium whose contents, when executed by a computing system, cause the computing system, to perform a method, the method comprising:
receive an indication of a drug or medication;
generate a shortage risk score for the drug or medication based on multiple factors; and
perform an action based on the generated shortage risk score.

14. The computer-readable medium of claim 13, wherein generating the shortage risk score includes:
inputting the multiple factors into a large language model (LLM);
generating a prediction for the shortage risk score.

15. The computer-readable medium of claim 13, wherein performing the action includes rendering a scoreboard of drugs or medications that presents the drugs or medications along with assigned generated risk scores.

16. The computer-readable medium of claim 13, wherein the shortage risk score for the drug or medication represents a predicted scarcity of the drug or medication based on the multiple factors.

17. The computer-readable medium of claim 13, wherein the multiple factors include drug age, brand/generic availability, pricing, associated recalls, number of manufacturers, and geographic locations of manufacturers.

18. The computer-readable medium of claim 17, wherein the multiple factors include disaster events at the geographic locations of the manufacturers and manufacturing events at the geographic locations of the manufacturers.

19. A method comprising:
accessing data from multiple, disparate, data sources;
applying a machine-learning (ML) transformer to the multiple, disparate data sources to identify at least one pair of interchangeable medications; and
performing an action based on the identification of the at least one pair of interchangeable medications.

20. The method of claim 19, wherein the multiple, disparate data sources include data sources associated with clinical trials and multiple hospital systems, and wherein the ML transformer generates a prediction of multiple characteristics shared by the at least one pair of interchangeable medications.
